(19) United States
(12) Patent Application Publication
(10) Pub. No.: US 2023/0346871 A1
(43) Pub. Date: Nov. 2, 2023

(54) COMPOSITIONS COMPRISING AQUEOUS OIL PALM FRUIT EXTRACT FOR INCREASING TYROSINE HYDROXYLASE LEVELS AND IMPROVING BRAIN HEALTH

(71) Applicant: MALAYSIAN PALM OIL BOARD, Kajang, Selangor (MY)

(72) Inventors: Vera V. KOLEDOVA, Medford, MA (US); Robert P. WEINBERG, Dixfield, MA (US); Anthony SINSKEY, Boston, MA (US); Chokyun RHA, Boston, MA (US); Kenneth C. HAYES, Wellesley, MA (US); Ravigadevi SAMBANTHAMURTHI, Kajang (MY); Kirsten C. SCHNEIDER, Somerville, MA (US); Anastasia A. ARTAMONOVA, Belmont, MA (US); Soon Sen LEOW, Kajang (MY)

(73) Assignee: MALAYSIAN PALM OIL BOARD, Kajang, Selangor (MY)

(21) Appl. No.: 17/782,953

(22) PCT Filed: Nov. 23, 2020
(86) PCT No.: PCT/MY2020/050165

§ 371 Date: Jun. 6, 2022

(30) Foreign Application Priority Data
Nov. 22, 2019 (MY) PI 2019006896

(51) Int. Cl. A61K 36/889; A61P 25/28; A23L 33/105; A23L 2/02

(52) U.S. Cl. CPC A61K 36/889; A61P 25/28; A23L 33/105; A23L 2/02

(57) ABSTRACT

The present invention discloses composition(s) for increasing the levels of tyrosine hydroxylase in the mammalian brain of human subjects and thus increasing the levels of brain catecholamines, in treating neurological related disorders. The composition(s) comprise(s) a biological extract obtained from the oil palm fruit bunch or aqueous stream of the processing or milling of oil palm fruit bunches. In an embodiment, the extract is obtained from the oil palm fruit bunch or aqueous stream of the processing or milling of oil palm fruit bunches. The extract is water-soluble. In another embodiment, the extract further comprises one or more organic compounds. The one or more organic compounds are selected from the group consisting of but not limited to sugars, phenolic compounds, flavonols, stilbenoids, dihydroflavonols, anthocyanins, shikimic acid, soluble fibres and peptides.

### FIELD OF INVENTION

The present embodiment generally relates to composition(s) for increasing tyrosine hydroxylase levels in treating neurological related disorders. More particularly, the present invention relates to composition(s) extracted from the aqueous stream of processing or milling of oil palm fruit bunches, also known as vegetative liquor, for increasing tyrosine hydroxylase levels that is useful in treating neurological disorders.

### BACKGROUND

Tyrosine hydroxylase is the rate-controlling enzyme in the biosynthesis of multiple catecholamines from the amino acid tyrosine. Catecholamines, including dopamine, epinephrine and norepinephrine, are important neurotransmitters in the brain and central nervous system. Catecholamines are also important hormones which circulate in the blood with specific functions in the autonomic nervous systems and sympathetic nervous systems mediating the physiological changes of the flight-or-fight response in many organisms.

Catecholamines are important in cognitive functioning, memory, attention, mood and multiple motor/sensory functions of the brain involved with vision, auditory circuits, speech, movement and communication. Relative deficits of catecholamines may cause several neurological and neurodegenerative conditions, where such deficits result in impaired neural circuitry.

Dopamine is a catecholamine which is a neurotransmitter in the brain that is utilised by brain neurons to transmit nerve impulses to control or modulate peripheral muscle movement. The degeneration of these dopamine-containing neurons results in lower levels of dopamine in the brain. Such levels disturb the normal neural circuits which depend on dopamine for normal muscle control and movement.

Treatment of neurological disorders, for example Parkinson's disease, is aimed towards controlling movement disorder and reducing a patient's symptoms primarily by the drug levo-3,4-dihydroxyphenylalanine (L-DOPA) which is subsequently metabolised to dopamine, or by administering drugs which directly stimulate the type D1 and D2 dopamine receptors.

Another exemplary drug treatment of Parkinson's disease involves the oral administration of L-DOPA, the hydroxylation product of tyrosine, which causes an increase in dopamine synthesis, as Parkinson's disease results from the deficiency of dopamine in the substantia nigra of the basal ganglia. L-DOPA is a pro-drug which is converted to dopamine in the brain.

Currently any relative or absolute deficiency of catecholamines in the brain may be treated by increasing the brain substrate level of the metabolic precursor to that deficit catecholamine, such as administering L-DOPA to patients to increase the decarboxylation to dopamine via Le Chatelier's principle, or else by administering an agonist ligand drug which may directly bind to the relevant catecholamine. Such drugs may have other side effects on a subject's body.

Accordingly, it would be desirable to have compounds, compositions and methods for treating neurological disorders that has minimal or zero side effects on a subject's body.

### SUMMARY OF THE INVENTION

In view of the foregoing, an embodiment herein provides composition(s) for increasing the levels of tyrosine hydroxylase in the mammalian brain of human subjects and thus increasing the levels of brain catecholamines, hence improving brain health. The composition disclosed herein is useful for the treatment of neurological disorders. The composition(s) include(s) a biological extract obtained from processing or milling of oil palm fruit bunches, also known as vegetation liquor. In an embodiment, the extract is obtained from aqueous stream of processing or milling of oil palm fruit bunches, also known as vegetation liquor. The extract is water-soluble. In another embodiment, the extract further includes one or more organic compounds. The one or more organic compounds are selected from the group consisting of but not limited to sugars, phenolic compounds, flavonols, stilbenoids, dihydroflavonols, anthocyanins, shikimic acid, soluble fibres and peptides.

In some embodiments, the extract further includes soluble or insoluble fibres. In some other embodiments, the extract further includes polymers or oligomers composed of monomers selected from the group consisting of but not limited to sugars, phenolic compounds, flavonols, stilbenoids, dihydroflavonols, anthocyanins, shikimic acid, soluble fibres and peptides. In another aspect, the composition(s) is/are nutraceutical or pharmaceutical composition(s). In an aspect, the composition(s) is/are administered orally or intravenously. In another embodiment, the extract further includes natural phenolic compounds.

In an aspect, a biological extract is obtained from processing or milling of oil palm fruit bunches for manufacturing composition(s) for treatment of a neurological or neurodegenerative conditions or disorders related to impairments of neurotransmitters in the brain and central nervous system.

In an aspect, the composition(s) is/are food composition(s) and dietary supplement(s). In another aspect, the composition(s) is/are used in combination with an edible or inedible oil. In another aspect, the composition(s) is/are a beverage. In yet another aspect, the composition(s) is/are nutraceutical or pharmaceutical composition(s).

These and other aspects of the embodiments herein will be better appreciated and understood when considered in conjunction with the following descriptions and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the concept thereof, and the embodiments herein include all such modifications.

### BRIEF DESCRIPTION OF THE DRAWINGS

Other objects, features and advantages of the embodiment will be apparent from the following descriptions when read with reference to the accompanying drawings. In the drawings, wherein like reference numerals denote corresponding parts throughout the several views:

FIG. 1 is a flowchart of a biosynthetic pathway for catecholamines;

FIG. 2 depicts 3-D protein conformation of tyrosine hydroxylase; and

FIGS. 3A-3H shows an experimental study of the effects of an aqueous extract obtained from the processing or milling of oil palm fruit bunches on rat brain, in accordance with an embodiment herein.

### DETAILED DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following descriptions.

Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practise the embodiments herein. Accordingly, the examples should not be construed as limiting the scope of the embodiments herein.

### Definitions

Palm oil is an edible vegetable oil derived from the mesocarp (reddish pulp) of the fruit of the oil palm. Palm oil mill defines a palm oil mill producing crude palm oil and kernels as primary products and biomass as secondary product. Aqueous defines a watery or water-soluble composition.

As mentioned, there remains a need for developing alternative organic composition(s) for treating neurological related disorders.

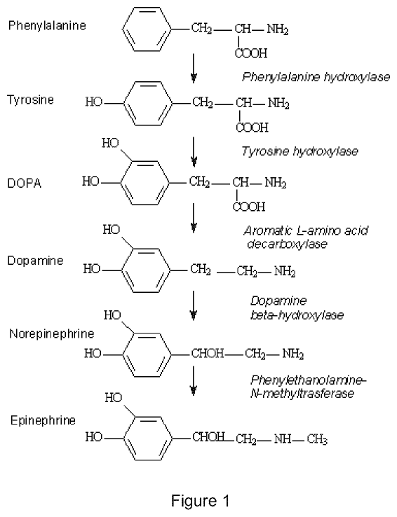

In an embodiment, the composition(s) include(s) a biological extract obtained from processing or milling of oil palm fruit bunches. For example, the extract is obtained from a fruit of the oil palm plant species Elaeis guineensis, Elaeis oleifera and/or interspecific hybrids of the two species. The extract is obtained from aqueous stream of processing or milling of oil palm fruit bunches. The extract is water-soluble. The extract is obtained from a vegetative liquor resulting from a palm oil milling process. The existing palm oil milling process involves palm oil processing, leading to two streams: aqueous and non-aqueous (oil stream). The aqueous stream is often ignored, considering no value thereof. However, the present embodiment opens the gateway for treating multiple diseases using the aqueous stream. The extract from the aqueous stream is configured for increasing the levels of tyrosine hydroxylase in the mammalian brain of human subject and thus increasing the levels of brain catecholamines, in treating neurological disorders. The brain catecholamines may include dopamine, norepinephrine and epinephrine. Tyrosine hydroxylase is the rate-controlling step in the biosynthesis of catecholamines in the mammalian brain. The catecholamine biosynthetic pathway (FIG. 1) involves the conversion of phenylalanine to tyrosine by hydroxylation.

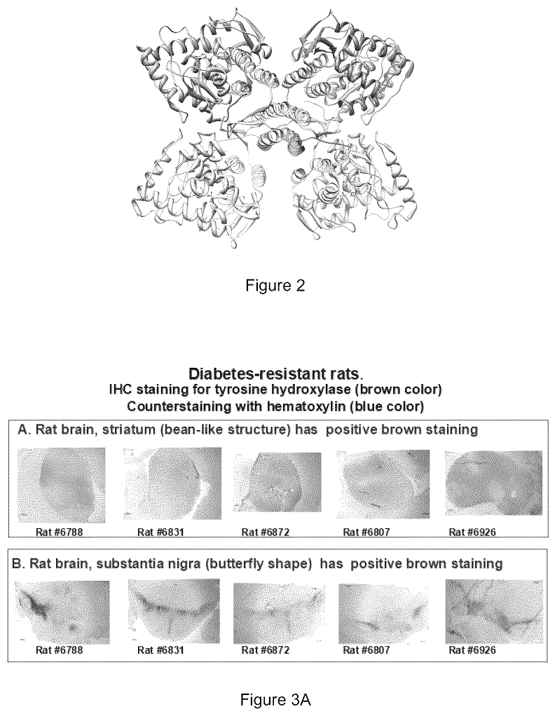

Tyrosine is then hydroxylated to DOPA by the enzyme tyrosine hydroxylase, 3D protein conformation thereof is shown herein in FIG. 2. DOPA is then decarboxylated to dopamine. Dopamine is then hydroxylated to norepinephrine. Finally norepinephrine is methylated to epinephrine.

In an embodiment, the extract further includes one or more organic compounds. The one or more organic compounds are selected from the group consisting of but not limited to sugars, phenolic compounds, flavonols, stilbenoids, dihydroflavonols, anthocyanins, shikimic acid, soluble fibres and peptides. In another embodiment, the extract further includes soluble or insoluble fibres.

In another embodiment, the extract further includes polymers or oligomers composed of monomers selected from the group consisting of but not limited to sugars, phenolic compounds, flavonols, stilbenoids, dihydroflavonols, anthocyanins, shikimic acid, soluble fibres and peptides.

In an embodiment, the extract further includes natural phenolic compounds. The natural phenolic compounds may further include cinnamate, benzoate, caffeate, coumarate, ferulate and sinapoate derivatives.

In an embodiment, the composition(s) is/are administered orally to human subjects.

In another embodiment, the composition(s) is/are administered intravenously. In an aspect, the composition(s) is/are in solid or pulverised form. In another aspect, the composition(s) is/are administered in liquid form. In another aspect, the composition(s) is/are administered in vaporised form or aerosol.

In an aspect, the composition(s) is/are nutraceutical composition(s). The composition(s) can be provided as a nutrition supplement to be consumed alone or supplemented with food or beverages or water. In another aspect, the composition(s) is/are pharmaceutical composition(s) in the form of tablets or liquid or gaseous. In some embodiments, the composition(s) may further include lipid-soluble compounds including tocotrienol, an essential fatty acid, an antioxidant, a peptide, a vitamin, or a mineral.

In an aspect, a biological extract is obtained from processing or milling of oil palm fruit bunches for manufacturing composition(s) for treatment of a neurological or neurodegenerative conditions or disorders related to impairments of neurotransmitters in the brain and central nervous system.

In another aspect, the subject has a genetic mutation in the catecholamine biosynthesis pathway. The mutation results in decreased hydroxylation of tyrosine.

The mutation may also lead to reduced levels of catecholamines in the brain. In some embodiments, the mutation may lead to relative or absolute deficiency of the level of dopamine, nonepinephrine or epinephrine in the brain. The mutation may also lead to Parkinsonian-like syndrome. In some other aspects, the mutation may also cause extrapyramidal pathology. In another aspect, the mutation may cause abnormal or ataxic movement disorder.

### EXPERIMENTAL STUDIES

The embodiment herein discloses the biological aqueous extract from the aqueous stream of processing or milling of oil palm fruit bunches being administered to 20 male Nile Grass rats of 3 weeks old. They were fed semipurified hiCHO diets (60:20:20, carbohydrate:fat:protein) with 10% of the extract or without the extract for 8 weeks. They were also observed by feeding the CHOW diets thereto without the extract until the development of severe diabetes. The groups were divided into diabetes susceptible or resistant based on RBG 75 mg/dl, or severe diabetes (CHOW diet) as shown in following table.

| PFJ supplementation | Diabetes status | n |
|---|---|---|
| No PFJ | Diabetes resistant | 5 |
| | Diabetes susceptible | 5 |
| | Severe diabetes | 3 |
| * PFJ 10% | Diabetes resistant | 4 |
| | Diabetes susceptible | 4 |

Further, formalin fixed brains were embedded in paraffin blocks and sliced into 8 μm sections. Staining was performed independently by Koch Institute Histology Core (blinded procedure) with primary anti-tyrosine hydroxylase antibodies (Novus Biologicals, cat. #NB300-109) at a dilution factor of 1:25.

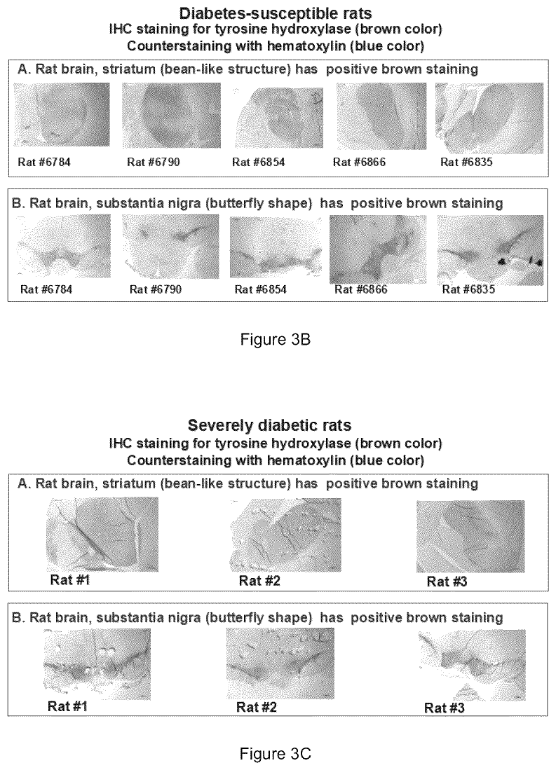

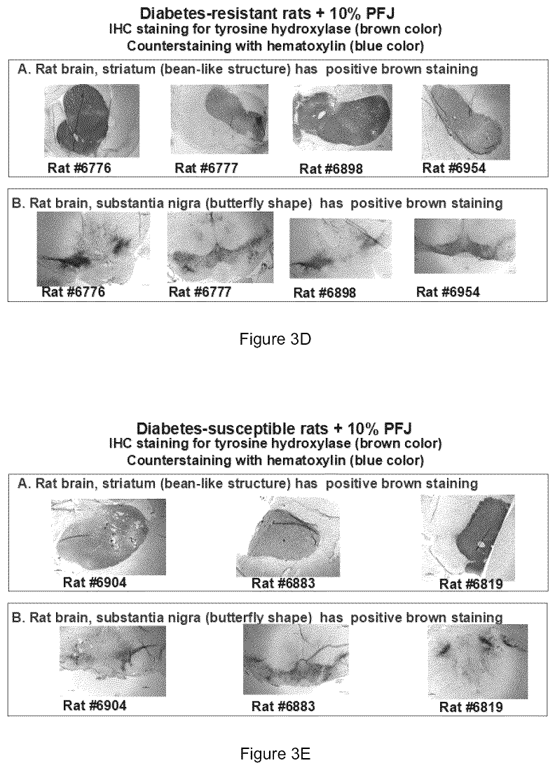

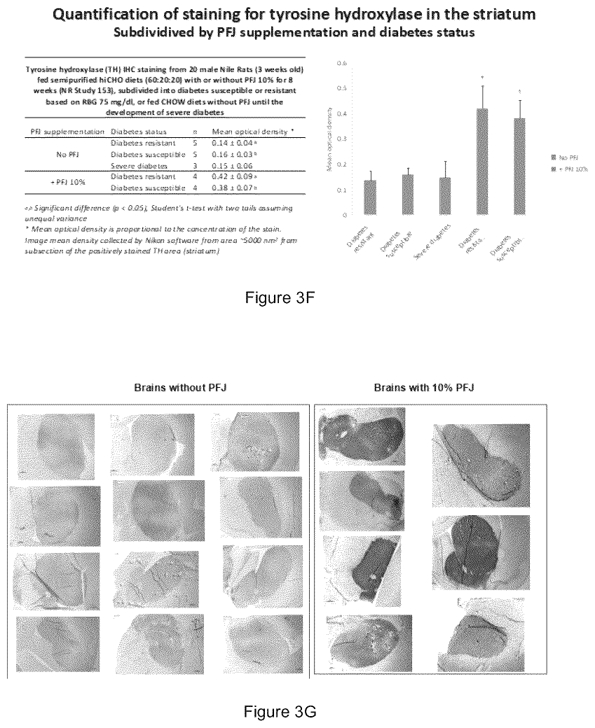

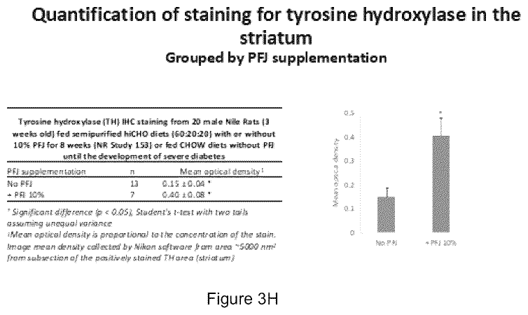

Images were acquired (as shown in FIGS. 3A, 3B, 3C, 3D, 3E, 3F, 3G, and 3H) by a Nikon transmitted light microscope at objective magnification 4×. Quantitative analysis was performed using Student's t-test and ANOVA.

As shown in FIG. 3F, PFJ (extract from the aqueous stream of processing or milling of oil palm fruit bunches is also referred to as Palm Fruit Juice, PFJ herein) supplementation increased the expression of tyrosine hydroxylase in the striatum of diabetes resistant rats by a factor 3 and diabetes susceptible rats by a factor of 2.4. As shown in the above figures, there are two regions in the Nile Grass rat brain which express tyrosine hydroxylase: Striatum and substantia nigra. There is no significant difference in the expression of tyrosine hydroxylase in the brains among diabetes resistant, diabetes susceptible or severely diabetic rats, without PFJ or the extract embodied herein. However, experimental studies prove that tyrosine hydroxylase expression in the Nile Grass rat's brain depends upon PFJ supplementation, which further opens the gateway for treating neurological related disorders and other disorders, but not the diabetes status.

As will be readily apparent to those skilled in the art, the present embodiment may easily be produced in other specific forms without departing from its essential characteristics. The present embodiment are, therefore, to be considered as merely illustrative and not restrictive, the scope being indicated by the claims rather than the foregoing descriptions, and all changes which come within therefore intended to be embraced therein.

1. A composition for use in increasing the levels of tyrosine hydroxylase in the mammalian brain of human subjects and thus increasing the levels of brain catecholamines, the composition;
a biological extract obtained from the oil palm fruit bunch containing organic compounds,
wherein said organic compounds include one or more of sinapoate derivatives, stilbenoids, phenolic compounds, flavonols, dihydroflavonols, anthocyanins, shikimic acids, soluble fibres, and peptides.

2. The composition for use of claim 1, wherein said extract further comprises natural phenolic compounds selected from the group consisting of cinnamate, benzoate, caffeate, coumarate, ferulate, and a combination thereof.

3. The composition for use of claim 1, wherein said composition further comprises an edible or an inedible oil.

4. The composition for use of claim 1, wherein the extract is obtained from aqueous stream of the processing or milling of oil palm fruit bunches.

5. The composition for use of claim 1, wherein the extract is water-soluble.

6. The composition for use of claim 1, wherein the extract further comprising soluble or insoluble fibres.

7. The composition for use of claim 1, wherein the composition is/are administered orally or intravenously.

8. The composition for use of claim 1, wherein the composition is/are nutraceutical or pharmaceutical compositions.

9. The composition for use of claim 8, wherein said nutraceutical composition is a beverage.

10. The composition for use of claim 8, wherein said pharmaceutical composition is in a form selected from the group consisting of solid form, pulverized form, tablets, liquids, gaseous, vaporized form, and aerosol form.

11. The composition for use of claim 1, further comprising lipid-soluble compounds selected from the group consisting of tocotrienols, essential fatty acids, antioxidants, peptides, vitamins, and minerals.

12. Use of a biological extract obtained from the aqueous stream of the processing or milling of oil palm fruit bunches for the manufacture of a medicament for the treatment of neurological or neurodegenerative conditions relating to decreased tyrosine hydroxylase levels in a subject's brain, or for controlling the rate of biosynthesis of catecholamines in the brain.

13. The use of a biological extract according to claim 12, wherein said catecholamines are selected from the group consisting of dopamine, norepinephrine, and epinephrine.

14. The use of a biological extract according to claim 12, wherein said biological extract further comprises polymers or oligomers composed of monomers selected from the group consisting of sugars, phenolic compounds, flavonols, stilbenoids, dihydroflavonols, anthocyanins, shikimic acid, soluble fibres, peptides, and a combination thereof.

15. The use of a biological extract according to claim 12, wherein said biological extract further comprises natural phenolic compounds selected from the group consisting of cinnamate, benzoate, caffeate, coumarate, ferulate, sinapoate derivatives, and a combination thereof.

16. The use of claim 12, wherein the composition is/are food composition and dietary supplements.

17. The use of claim 12, wherein the composition is/are used in combination with an edible or inedible oil.

18. The use of claim 12, wherein the composition is/are a beverage.

19. The use of claim 12, wherein the composition is/are nutraceutical or pharmaceutical compositions.
